(19) United States
(12) Patent Application Publication
(10) Pub. No.: US 2014/0275233 A1
(43) Pub. Date: Sep. 18, 2014

(54) ACTIVATED SOY POD FIBER

(71) Applicant: MicroBiome Therapeutics LLC, Broomfield, CO (US)

(72) Inventor: Mark L. Heiman, Indianapolis, IN (US)

(21) Appl. No.: 14/206,674

(22) Filed: Mar. 12, 2014

Related U.S. Application Data
(60) Provisional application No. 61/789,614, filed on Mar. 15, 2013.

(51) Int. Cl. A61K 31/352; A61K 36/48

(52) U.S. Cl. CPC A61K 31/352; A61K 36/48
USPC 514/453; 549/382

(57) ABSTRACT

The present invention provides, among other things, compositions and methods of manufacturing edible soy pod fiber comprising glyceollins. In some embodiments, methods of treating overweight, obesity, prediabetes, diabetes, or gastrointestinal dysbiosis in a subject are provided comprising orally administering a composition or food product comprising edible soy pod fiber comprising glyceollins.

### CROSS REFERENCE TO RELATED APPLICATIONS

This application claims priority to U.S. Provisional Application No. 61/789,614, filed Mar. 15, 2013, the entire contents of which are hereby incorporated by reference in their entirety for all purposes.

### BACKGROUND

One of the benefits of modern technology is that human longevity and health have improved, but modern society promotes more sedentary occupations. Before industrialization and the transition from farm to metropolis, the human body did the work.

Modern agricultural systems are developed with two related goals in mind: to obtain the highest yields possible and to get the highest economic profit possible. Benefits of food processing include preservation, efficient marketing and distribution, and increasing food consistency. In addition, it increases yearly availability of many foods, enables transportation of delicate perishable foods across long distances and makes many kinds of foods safe to eat by de-activating spoilage and pathogenic micro-organisms. An unfortunate consequence of modern food processing is that more calories are packed into a gram of food than ever before.

As a consequence of modernization, people of modern societies live longer, consume more calories in a day and are more sedentary. There is a great risk that loss of metabolic fitness accompanies modernization. Each cell of the body has an important function and it consumes energy to perform that function. The energy is supplied by metabolism of food and often measured by heat units or calories. Energy balance is a point when the total energy supplied by diet matches the total energy demand of all cells. To prepare for periods of negative energy balance or when the total energy spent is greater than total energy consumed, energy is convened to fat and it is stored in fat cells or adipocytes. Storage occurs during periods of positive energy balance or when total calories consumed and absorbed are greater than total calories utilized during that period. A metabolically fit individual is one who consumes sufficient calories to meet the energy demand and deposit excess calories as fat in adipocytes. However, a consequence of modernization is often a loss of metabolic fitness. There is such an abundance of calories consumed that the adipocytes become overloaded and fat synthesized for storage is hoarded in other tissues.

While it is perfectly healthy to store fat in adipocytes, it is unhealthy to store fat in any other organ like liver, arteries, pancreas, muscle, bone, brain, etc. Modernization has resulted in discovery of new methods to measure the level of fat supply and to locate where fat is being accumulated. An inexpensive method is to measure height and body weight to calculate a body mass index or BMI. It appears one is metabolically fit when BMI is about 25. But a loss of fitness is observed when BMI is greater than 25. Obesity is defined when BMI is 30 or greater. It is the consequence of fat stored in tissues other than adipocytes that is unhealthy and there are numerous studies using BMI as an index to monitor and correlate many pathological findings, such as diabetes and cardiovascular diseases.

Diabetes affects nearly 25.8 million people or 8.3% of the U.S. population and is projected to rise significantly over the next decade. In most cases, the diabetes results from excess fat stored in tissues that utilize insulin to supply them with energy (glucose) needed for their specialized function. Those tissues become increasing less sensitive to insulin as they accumulate fat and glucose remains in the blood. This is termed type 2 diabetes (T2D). The global incidence of T2D is at a pandemic rate as more societies become modernized. Diabetes is the seventh leading cause of death in the U.S. In addition to these staggering mortality data, diabetes results in devastating morbidities that result in high healthcare costs. Indeed, after adjusting for population, age, and sex differences, average medical expenditures among people with diabetes were 2.3-times higher than what expenditures would be in the absence of diabetes. Notably, a 2007 estimate suggests that the total (direct and indirect) estimated cost for diabetes was $174 billion in that year alone.

In order to better educate non-diabetic patients about their potential for progressing toward a clinical case of diabetes, the Centers for Disease Control (CDC) and the American Diabetes Association (ADA) coined the term “prediabetes”. In this way, medical practitioners can identify patients at higher risk for developing diabetes. Those patients typically have a BMI between 25 and 35 and T2D is closely associated with BMIs between 30 and 45. The CDC estimates that there are 79 million Americans aged 20 years or older with prediabetes. Without intervention, about 11% are expected progress to type 2 diabetes (T2D) in just 3 years. Prediabetes is defined by the ADA as fasting blood glucose levels between 100 mg/dl and 125 mg/dl, or blood glucose level between 140 mg/dl and 125 mg/dl 2 h after an oral glucose tolerance test (OGTT) and a hemoglobin A1c level between 5.7% and 6.4%. Data exist to support that targeted treatment regimens for prediabetics can significantly reduce the risk of progressing to T2D. For example, the Diabetes Prevention Program (DPP) demonstrated that prediabetics who received intensive counseling on diet, exercise, and behavior modification were able to reduce their risk of developing diabetes by 58 percent and those who took metformin reduced the risk of developing diabetes by 31 percent (N Engl J Med, 2002, 346:393-403). Moreover, if reversion to normal glucose regulation occurred only transiently, there was a significantly reduced risk of progressing to diabetes. Thus, there is a need for additional prediabetes interventions that are inexpensive, safe, and efficacious.

At the end of 2007, the US National Institutes of Health (NIH) launched the Human Microbiome Project (HMP) and, in early 2008, the European Commission and China initiated the Metagenomics project of the Human Intestinal Tract (MetaHIT). These large efforts apply advanced sequencing and bioinformatic tools to characterize the microbes living in and on our bodies. An estimated 100 trillion microorganisms reside in the large intestine where they play a role in metabolizing food and converting it to energy for cellular work or to be banked in reserve. Understanding how the activities of these microbial populations impact human metabolism may offer approaches to develop interventions to prevent metabolic unfitness and to treat obesity and diabetes.

For example, Chinese T2D patients have recently been characterized with a moderate degree gastrointestinal (GI) bacterial dysbiosis (Nature, 2012, 490:55-60) or an abnormal population of microbiota. Analysis reveals that the GI microbiota of those T2Ds have decreased ability to synthesize short chain fatty acids (SCFAs), an increased ability to produce hydrogen sulfide, an increased ability to produce methane, and decreased defense against oxidative stress. A metabolically unfit population would benefit from supplementing their diets with poorly absorbed antioxidants. One class of antioxidant polyphenolics called isoflavones is produced by soy plants and is shown to promote health in humans. For example, the isoflavones genistein, daidzein, and glycitein are in particularly high levels in traditional soy-based foods. Consumption of a diet rich in soy products may prevent T2D. Other isoflavones are induced by the plant's defense mechanisms. Those compounds are termed phytoalexins. Three very similar phytoalexins called glyceollin I, glyceollin II, and glyceolin III, are produced by soy when the plant is exposed to soil microorganisms, ultraviolet (UV) light or heavy metals (J. Agric. Food Chem., 2009, 57: 2614-2622) and are very potent antioxidants J. Agric. Food Chem 2010; 58: 11633-11638). Supplementation of the prediabetic's diet with the glyceollins will deliver an antioxidant to the GI biome. Without wishing to be bound by theory, it is believed herein that changing the redox potential of the GI biome that selects for families of microbiota will benefit the prediabetic.

### SUMMARY

The present invention provides, among other things, compositions comprising isolated plant tissue having a high glyceollin content per gram of plant tissue.

In some embodiments, the isolated plant tissue has a glyceollin content of at least 0.1, 0.15, 0.2, 0.25, 0.3, 0.35, 0.4, 0.45, or 0.5 mg per gram of plant tissue. In some embodiments, the plant tissue comprises soy pod tissue.

In some embodiments, a composition comprising isolated soy pod tissue containing one or more glyceollins is provided. In some embodiments, the combined total content of one or more glyceollins in the soy pod tissue is at least 1, 5, or 10 mg per gram.

In some embodiments, isolated soy pod containing both soluble and insoluble dietary fiber is provided.

In some embodiments, isolated soy pod formulated for oral delivery is provided.

In some embodiments, the invention provides a food product comprising dietary fiber from soy pod tissue. In some embodiments, the food product comprises one or more glyceollins. In some embodiments, the food product comprises glyceollins in a total amount of at least 25, 50, 75, 100, 200, or 250 mg.

In some embodiments, the invention provides a powder comprising one or more glyceollins. In some embodiments, the powder is made from soy pod tissue. In some embodiments, the powder comprises one or more glyceollins at a combined total content of at least 1, 2.5, 5, 7.5, 10, or 15 mg glyceollins per gram of powder.

The invention further provides methods for treating a subject suffering from or susceptible to overweight or obesity. In some embodiments, the methods comprise orally administering to the subject a composition or food product as described herein.

The invention further provides methods for treating subject suffering from or susceptible to diabetes, or prediabetes. In some embodiments, the methods comprise orally administering to the subject a composition or food product as described herein.

The invention further provides methods for modifying the gastrointestinal microbiome of a subject, wherein the gastrointestinal microbiome of the subject includes a first population of bacteria that process fat and protein, and a second population of bacteria that ferment carbohydrate and produce increases in small chain fatty acids. In some embodiments, the method comprises administering to the subject a composition comprising an effective amount of one or more glyceollins to shift the relative abundance of the first population of bacteria and the second population of bacteria in the gastrointestinal tract.

In some embodiments, the first population comprises the genus of Ruminococcaceae and the second population comprises the genus of Blautia.

In some embodiments, methods are provided for modifying the level of Blautia in the microbiota taxa of a subject. In some embodiments, a subject is identified as having Blautia level below 2, 3, 4, or 5% abundance and in need of treatment with an effective amount of one or more glyceollins to increase Blautia levels to at least 10%, 15%, 20%, 25%, or 30% abundance. In some embodiments, a subject identified as in need of treatment is administered a composition comprising one or more glyceollins to increase Blautia levels.

In some embodiments, methods for treating gastrointestinal dysbiosis are provided comprising the step of orally administering to the subject an effective amount of a composition comprising one or more glyceollins.

The invention further provides methods of manufacturing a powder comprising soy pod dietary fiber and one or more glyceollins. In some embodiments the method comprises the steps of obtaining soy pod tissue, slicing or mincing the soy pod tissue, drying the soy pod tissue, and pulverizing the soy pod tissue into a powder. In some embodiments, the method comprises adding one or more glyceollins to the soy pod tissue. In some embodiments, the method comprises exposing the soy pod tissue to ultraviolet radiation.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The following figures are presented for the purpose of illustration only, and are not intended to be limiting.

FIG. 1 shows exemplary results illustrating plasma levels of glyceollins in ZDSD/Pco rats after administration of glyceollins (30 and 90 mg/kg, p.o.). Values represent the mean±SEM from 3 different rats at each time point and dose.

FIG. 2 shows exemplary results illustrating blood glucose levels of prediabetic ZDSD/Pco rats after administration of glucose (2 g/kg, p.o., at time 0). Glyceollins were administered (30 and 90 mg/kg, p.o.) 1 h prior to the start of the oral glucose tolerance test. Each symbol represents the mean±SEM of the blood glucose value for 8 rats. At 60 min, the blood glucose levels for the glyceollin treated animals were significantly lower than those for the vehicle-treated rats, and the areas under the curves for the glyceollin groups were significantly less than that integrated for the vehicle-treated rats.

FIG. 3 shows exemplary results illustrating insulin-mediated glucose uptake by 3T3-L1 adipocytes. Cells were exposed to insulin for 30 min at 37° C. followed by 10 min of incubation with [³H]-2Deoxy-glucose. The effective concentration for 50% increase in glucose uptake (EC₅₀) was 1.92 nM when computed by the 4-parameter logistic equation using SigmaPlot. These data are the average of 6 experiments that were normalized by calculating the percent cpm glucose uptake compared to basal cpm glucose uptake. The symbols represent mean±SEM and the line represents the best fit to the data using the 4-parameter logistic equation.

FIG. 4 shows exemplary results illustrating insulin, glyceollins, and insulin combined with glyceollins stimulated glucose uptake by 3T3-L1 adipocytes. Adipocytes were exposed to inulin, glyceollins, or both for 3 h. These data are the average of 3 experiments that were normalized by calculating the percent cpm glucose uptake compared to basal cpm glucose uptake. The symbols represent mean±SEM. All means for insulin-stimulated glucose uptake with different letters are significantly (p<0.05) different.

FIG. 5 shows exemplary results illustrating glyceollin-mediated glucose uptake by 3T3-L1 adipocytes. Cells were exposed to glyceollin for 45 min at 37° C. followed by 10 min of incubation with [³H]-2-deoxy-glucose. The EC₅₀ was 2.40±0.43 μM and a maximal uptake of 2.04±0.24-fold (computed by the 4-parameter logistic equation). These data are the average of 3 experiments that were normalized by calculating the percent cpm glucose uptake compared to basal cpm glucose uptake. The symbols represent mean±SEM and the line represents the best fit to the data using the 4-parameter logistic equation.

FIG. 6 shows exemplary results illustrating glyceollins stimulate the expression of glucose transporter genes GLUT1 and GLUT4 in 3T3-L1 adipocytes. mRNA levels of both genes were measured by real time PCR and are shown relative to mRNA level of RPL32. The cells were exposed to glyceollin for 3 h, mRNA was isolated from the cells, cDNA was synthesized, and gene expression was quantitated by real time PCR. Symbols represent mean±SEM.

FIG. 7 shows exemplary results illustrating daily administration of a glyceollin blend (90 mg/kg, p.o.) decreases fat mass of prediabetic rats by 11 days. Fat mass was measured by quantitative NMR.

FIG. 8 shows exemplary results illustrating daily administration of the glyceollin blend (90 mg/kg, p.o.) decreases plasma leptin and tends to increase plasma GLP-1 of prediabetic rats by 11 days. Plasma hormones were measured in trunk blood by ELISA at the end of the study.

FIG. 9 shows exemplary results illustrating daily administration of the glyceollin blend (90 mg/kg, p.o.) increases plasma insulin of prediabetic rats by 11 days. Plasma insulin was measured in trunk blood by ELISA at the end of the study and plasma glucose was measured by glucometer.

FIG. 10 shows exemplary results illustrating an HPLC chromatogram revealing compounds present in soy powder after 0 to 72 incubation following 2 minutes exposure to ultraviolet-B radiation.

FIG. 11 shows exemplary results illustrating glyceollin content of soy pod after 0 to 96 hours of incubation following slicing into 1 mm cross sections and 2 minutes of exposure to ultraviolet-B radiation.

### DEFINITIONS

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Amelioration: As used herein, the term “amelioration” means the prevention, reduction or palliation of a state, or improvement of the state of a subject. Amelioration includes, but does not require, complete recovery or complete prevention of a disease condition.

Comparable: As used herein, the term “comparable” refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

Correlates: As used herein, the term “correlates”, refers to its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

Dysbiosis or Gastrointestinal dysbiosis: As used herein, the term “dysbiosis” (also called dysbacteriosis) as used herein refers to a condition when a microbial population occupying a habitat on or in the body during health is shifted to a population of microbiota identified in the same habitat in an unhealthy or diseased state. Dysbiosis is most prominent in the digestive tract (also called gastrointestinal dysbiosis) where it is associated with illnesses such as diabetes, obesity, irritable bowel syndrome, inflammatory bowel disease and gastric ulcers.

Food product: As used herein, the term “food product” refers to food or a food ingredient that is specially formulated and intended for the dietary management of a disease that has distinctive nutritional needs that cannot be met by normal diet alone.

Glyceollins: As used herein, the term “glyceollins” refers to the phytoalexins glyceollin I, glyceollin II, and glyceolin III, and similar compounds that are potent antioxidants produced in soy when the plant is exposed to soil microorganisms, ultraviolet (UV) light or heavy metals. Phytoalexins are isoflavones that are induced by a plant's defense mechanisms.

Improve, increase, or reduce: As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with overweight, obesity, prediabetes, diabetes, or gastrointestinal dysbiosis, who is about the same age and/or gender as the individual being treated (to ensure that the stages of the disease in the treated individual and the control individual(s) are comparable).

Microbiome or Gastrointestinal microbiome: As used herein, the term “microbiome” refers to the totality of microbes, their genetic elements (genomes), and environmental interactions in a particular environment (habitat or ecosystem). The term “gastrointestinal microbiome” refers to the microbiome of the gastrointestinal tract.

Prediabetes: As used herein, the term “prediabetes” refers to a condition in which individuals have fasting blood glucose or hemoglobin A1c levels higher than normal but not high enough to be diagnosed as diabetic. People with prediabetes have an increased risk of developing type 2 diabetes.

Providing: As used herein, the term “providing” refers to performing a manipulation that causes an entity of interest to be present at a level and/or with an activity higher than that observed under otherwise comparable conditions prior to or absent the manipulation. In some embodiments, providing consists of or comprises administering the entity itself (alone or as part of a composition); in some embodiments, providing consists of or comprises administering an agent that causes an increase in level and/or activity of the entity of interest.

Reference: A “reference” entity, system, amount, set of conditions, etc., is one against which a test entity, system, amount, set of conditions, etc. is compared as described herein. For example, in some embodiments, a “reference” individual is a control individual who is not suffering from or susceptible overweight, obesity, diabetes, or gastrointestinal dysbiosis; in some embodiments, a “reference” individual is a control individual afflicted with the same form of disease as an individual being treated, and optionally who is about the same age as the individual being treated (to ensure that the course of the disease or pre-diseased state in the treated individual and the control individual(s) are comparable).

Subject: As used herein, the term “subject”, “individual”, or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.). In some embodiments, the subject to be treated is an individual (infant, child, adolescent, or adult human) having or having the potential to develop overweight, obesity, diabetes, or gastrointestinal dysbiosis. In some instances, a subject to be treated is genetically predisposed to developing overweight, obesity, diabetes, or gastrointestinal dysbiosis.

Therapeutic agent: As used herein, the phrase “therapeutic agent” refers to any agent that, when administered to a subject, has a therapeutic effect and/or elicits a desired pharmacological and/or biological effect.

Therapeutic regimen: As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease. In some embodiments, treatment includes administration of one or more therapeutic agents either simultaneously, sequentially or at different times, for the same or different amounts of time.

Therapeutically effective amount: As used herein, the term “therapeutically effective amount” refers to an amount of a therapeutic agent (e.g., an edible fiber comprising glyceollins) which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the particular form of overweight, obesity, diabetes, or gastrointestinal dysbiosis being treated; the severity of the condition or pre-condition; the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

Treatment: As used herein, the term “treatment” (also “treat” or “treating”) refers to any administration of a therapeutic agent (e.g., an edible fiber comprising glyceollins) according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., overweight, obesity, prediabetes, diabetes, gastrointestinal dysbiosis); in some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION

The present disclosure encompasses the findings that edible fiber can be produced from soy plant tissue and food products containing edible fiber enhanced with glyceollins is useful for the treatment or prevention of overweight, obesity, prediabetes, diabetes, and gastrointestinal dysbiosis.

Edible Fiber Produced from Soy Plant

Most soy products are prepared from soybeans (the soy seeds contained in the soy pod. Those products are derived from soy oil and soy protein in the soybeans. Common products are soy sauce, soy oil, soy milk, and tofu. The shell of the soybean pod is the ovary wall. This protects the ovules (seeds or beans) and provides a safe environment for them to grow and mature. Soy pods are dehiscent, meaning they have a seam that runs along both sides that can split open. The inside of a soy pod is known as the locule. Other than edamame, there are currently no edible products produced from the pod. Further, most consumers do not eat the pods when served unshelled edamame (pods with beans). It is more popular to serve shelled edamame or the soybeans, which are eaten uncooked or after cooking. Edamame is a variety of soy that is engineered to offer tasty large beans when picked during the middle stages of the bean growth and maturity. However, for the production of edible fiber as described herein, any variety of soy can be used when the bean is harvested at a middle reproductive stage.

### Enhancing Glyceollin Content of Soy Plant Tissue

The soy pod does not synthesize glyceollin unless it is exposed to an environmental stressor. Exposure to UV light offers an efficient elicitor of glyceollin by increasing the expression of polyphenylalanine ammonia-lyase and chalcone synthase. UV photoactivation lends itself to large scale low cost development of a marketed product. Additionally, or alternatively, glyceollin may be elicited by slicing or mincing.

Pods are be detached from the plant and opened at the seam to remove them from the seeds. Soy pods will typically be harvested at reproductive stage R6. This stage contains green seeds that fill the pod cavity and is the stage that edamame is harvested. This is a good source of edible fiber because pods contain bioactive isoflavones and they contain a blend of soluble and insoluble fiber. Moreover, soy pods are capable of producing the glyceollins when exposed to UV light and pods are of low economic value when edamame seeds are harvested. Dietary soy pod fiber is produced by milling.

Soy pods may be separated into halves after removing the beans and in some instances each half maybe cut into sections ranging from 0.5 cm to 2 cm. In some instances the entire pod with bean may be processed in small sections or slices by a food processor. In other instances, the pods maybe opened to harvest the beans and the pods can be sliced into small sections with a food processor.

Pods, cut pods, or sliced pods can optionally be irradiated using an ultraviolet (UV) light system producing UV-B light. Plant tissue will be arranged to expose one surface facing the lamp for 30-120 seconds and the tissue will be inverted to expose the other side for an additional 30-120 seconds. Photoactivated soy pod tissues can be placed at room temperature in a humidified chamber (45% to 85% humidity) in the dark for 24-72 h to permit the glyceollins to accumulate.

Soy isoflavones can be extracted with methanol and analyzed by HPLC to measure the extent of photoactivation. Daidzin, genistin, malonyldaidzin, malonylgenistin, daidzein, genistein, coumestrol, glyceollin III, glyceollin II, and glyceollin I can be measured.

If the desired isoflavone content of the activated fiber is not achieved under optimal conditions, an aliquot of the blend can be extracted with ethanol to concentrate the isoflavones into a stock solution so that the fiber can be spiked to contain the desired target content of isoflavones in the powdered fiber.

### Production of Powder

Edible or dietary fiber is defined as the remnants of plant components resistant to hydrolysis by human alimentary enzymes which include non-starch polysaccharides, resistant starch and lignin. Edible fiber is typically isolated from oats, barley, chicory roots, and sugar beets. Prior to this disclosure, dietary fiber has not been developed from soy pods. The plant material after the photoactivation is dried using a freeze dryer for 8-12 hours. The dried material can then be ground to a fine powder using a mill with a screen sifting particles between 0.5 mm and 1.5 mm. This milled material contains both soluble and insoluble fibers.

### Oral Administration

It is preferred that the compositions described herein be consumed orally so that the fiber enters the digestive tract. That will permit the fiber to interact with the microbiota that are resident in the lower GI tract. Some of the bacteria will thrive on the fiber and produce healthy byproducts such as small chain fatty acids that can be absorbed into the blood, serve as nutrients for the intestines, and serve as substrates for other bacteria. The novel fiber will also alter the redox potential of the intestinal milieu, which will aid in selection of desired species of healthy microbiota and in shifting the GI microbiome from an unhealthy state to one promoting heath.

Select diets, foods, food ingredients and other compositions comprising glyceollins all have the potential to interact and modify the GI microbiome if they are ingested, not metabolized by the digestive system and not absorbed by the intestines.

A therapeutically effective amount of the compositions described herein is largely determined based on the total amount of edible fiber and/or glyceollins contained in the food products described herein. Generally, a therapeutically effective amount is sufficient to achieve a meaningful benefit to a subject (e.g., treating, modulating, curing, preventing and/or ameliorating overweight, obesity, diabetes, or gastrointestinal dysbiosis).

In some embodiments, a therapeutically effective amount ranges from about 0.005 mg/kg body weight to 15 mg/kg body weight, e.g., from about 0.005 mg/kg body weight to about 12 mg/kg body weight, from about 0.005 mg/kg body weight to about 10 mg/kg body weight, from about 0.005 mg/kg body weight to about 5 mg/kg body weight, from about 0.005 mg/kg body weight to about 1 mg/kg body weight, from about 0.01 mg/kg body weight to about 15 mg/kg body weight, from about 0.01 mg/kg body weight to about 10 mg/kg body weight, from about 0.01 mg/kg body weight to about 5 mg/kg body weight, from about 0.01 mg/kg body weight to about 1 mg/kg body weight, from about 0.1 mg/kg body weight to about 15 mg/kg body weight, from about 0.1 mg/kg body weight to about 10 mg/kg body weight, from about 0.1 mg/kg body weight to about 2 mg/kg body weight, from about 0.1 mg/kg body weight to about 1 mg/kg body weight, from about 1 mg/kg body weight to about 15 mg/kg body weight, from about 5 mg/kg body weight to 15 mg/kg body weight, or from about 5 mg/kg body weight to 10 mg/kg body weight.

In some embodiments, a therapeutically effective dose is greater than about 0.0001 mg/kg body weight, greater than about 0.0005 mg/kg body weight, greater than about 0.001 mg/kg body weight, greater than about 0.005 mg/kg body weight, greater than about 0.01 mg/kg body weight, greater than about 0.05 mg/kg body weight, greater than about 0.1 mg/kg body weight, greater than about 0.5 mg/kg body weight, greater than about 1 mg/kg body weight, greater than about 5 mg/kg body weight, greater than about 10 mg/kg body weight, or greater than about 15 mg/kg body weight.

In some embodiments, a therapeutically effective dose can be expressed as an amount per unit volume. It is to be further understood that for any particular subject, specific dosage regimens can be adjusted over time according to the individual need and that dosage ranges set forth herein are exemplary only and are not intended to limit the scope or practice of the claimed invention.

### Therapeutic Uses

The present invention encompasses the surprising finding that oral administration of food products comprising glyceollins are useful, among other things, in the treatment or prevention (i.e., delay of onset) of overweight, obesity, prediabetes, diabetes, and gastrointestinal dysbiosis.

### Treatment of Overweight or Obesity

In certain embodiments, treatment of overweight or obesity refers to partial or complete alleviation, amelioration, relief, inhibition, delaying onset, reducing severity and/or incidence of symptoms.

Obesity is a complex, multi-factorial chronic disease involving environmental (social and cultural), genetic, physiologic, metabolic, behavioral and psychological components. It is the second leading cause of preventable death in the United States. Obesity increases the risk of developing hypertension, type 2 diabetes, stroke, gallbladder disease, infertility, osteoarthritis, sleep apnea, and cancer of the breast, prostate and colon. Persons with obesity may also be victims of employment and other discrimination and are penalized for their condition despite many federal and state laws and policies.

If maintained, even weight losses as small as 10 percent of body weight can improve the risk of developing the above diseases. In particular, hypertension, control of blood glucose and sleep apnea are improved with fat loss.

One goal in obesity treatment is to reduce excess fat storage. More specifically, to reduce extra-adipose fat stores. This may be measured by instruments using x-ray technologies, magnetic resonance technologies, and volume displacement technologies. More simply, it may be measured simply by measuring body weight, skin fold thickness and waist circumference. Sometimes an index of improvement is observed by changes in biomarkers such as a decrease in blood lipids, increased insulin sensitivity, decrease in circulating liver enzymes, decrease in leptin, increase in adiponectin and a decrease in markers of inflammation.

### Treatment of Diabetes and Related Disorders

In certain embodiments, treatment of diabetes or prediabetes refers to partial or complete alleviation, amelioration, relief, inhibition, delaying onset, reducing severity and/or incidence of symptoms.

Glucose comes from the food you eat and is also produced by liver and skeletal muscles. Insulin is a hormone, made by the pancreas and is released into the blood when glucose levels rise. Insulin transports glucose from the blood into cells of tissues to be used for energy. If insulin levels released are too low, or if the cells are resistant to insulin, glucose can't enter certain cells and remains in the blood. Blood glucose levels rise and are used to diagnose prediabetes or diabetes.

Prediabetes is defined by the American Diabetes Association as fasting blood glucose levels between 100 mg/dl and 125 mg/dl, or blood glucose level between 140 mg/dl and 125 mg/dl 2 h after an oral glucose tolerance test (OGTT) and a hemoglobin A1c level between 5.7% and 6.4%.

Type 1 diabetes, formerly called juvenile diabetes or insulin-dependent diabetes, is usually first diagnosed in children, teenagers, or young adults. With this form of diabetes, the pancreas no longer makes insulin because the body's immune system has attacked and destroyed the insulin producing cells. Treatment for type 1 diabetes includes insulin injections.

Type 2 diabetes, formerly called adult-onset diabetes or noninsulin-dependent diabetes, is the most common form of diabetes. People can develop type 2 diabetes at any age—even during childhood. This form of diabetes usually begins with insulin resistance, a condition in which fat, muscle, and liver cells do not use insulin properly. At first, the pancreas keeps up with the added demand by producing more insulin. In time, however, it loses the ability to secrete enough insulin in response to meals. Being overweight and inactive increases the chances of developing type 2 diabetes.

Some women develop gestational diabetes during the late stages of pregnancy. Although this form of diabetes usually goes away after the baby is born, a woman who has had it is more likely to develop type 2 diabetes later in life.

Symptoms of diabetes include increased thirst, frequent urination, frequent infections, blurred vision, feeling tired, slow wound healing, tingling and (or) numbness in the hands and (or) feet, and recurring skin, gum, or bladder infections, weight loss, nausea, and vomiting. If not treated the patients are at greater risk for many additional ailments.

People with diabetes are at increased risk for eye complications such as retinopathy. Diabetics are also at increased risk of nerve damage. Ulcers occur most often on the ball of the foot or on the bottom of the big toe. As many as 2 out of 3 adults with diabetes have high blood pressure. Hearing loss is twice as common in people with diabetes as it is in those who don't have the disease. Research shows that there is an increased prevalence of gum disease among those with diabetes, Gastroparesis is a disorder affecting people with both type 1 and type 2 diabetes in which the stomach takes too long to empty its contents (delayed gastric emptying). Diabetes can damage the kidneys and cause them to fail. Two out of 3 people with diabetes die from stroke or heart disease.

Improvement in diabetes is typically measured by analyzing blood glucose levels during fasting, after meals, after ingestion of a glucose drink and before bedtime. Lower fasting glucose levels and a more rapid and complete return to baseline glucose values after a meal or oral glucose challenge serve as indications of improvement.

### Treatment of Gastrointestinal Dysbiosis

In certain embodiments, treatment of gastrointestinal dysbiosis refers to partial or complete alleviation, amelioration, relief, inhibition, delaying onset, reducing severity and/or incidence of symptoms.

The GI microbiome may be characterized in healthy individuals and those inflicted with disease. In healthy individuals the GI microbiome is defined as normal. The GI microbiome characterized in those with certain diseases such as diabetes, obesity, irritable bowel syndrome (IBS) and irritable bowel disorder (IBD) are referred to as being in a state of dysbiosis. Currently, the symptoms and consequences of the pathological states define the diseases. It is unknown to what extent the dysbiosis contributes to the pathology or to what extent the dysbiosis is a consequence of that pathology. Nonetheless, the pathology or consequences thereof may be treated by converting the dysbiosis back to a normal GI microbiome.

GI dysbiosis is typically characterized as the microbiota community in a stool sample of an individual in a pathological state. In some cases, the dysbiosis results in reduced levels of SCFAs in the stool, increased fecal pH, increased production of hydrogen sulfide and methane gases, reduced antioxidant capacity, presence of opportunistic microbiota, presence of pathogenic fungi and yeast, increased intestinal inflammation, decreased intestinal mucosal thickness, colon ulcers and leaky gut. Improvements may be observed from increased SCFA levels in stool, decreased fecal pH, decreased production of hydrogen sulfide and methane gases, increased antioxidant capacity, absence of opportunistic microbiota, absence of pathogenic fungi and yeast, decreased intestinal inflammation, normal intestinal mucosal thickness, healthy colon anatomy and less circulating immunoglobulin A antibodies.

### EXAMPLES

The invention is further illustrated by the following examples. The examples are provided for illustrative purposes only. They are not to be construed as limiting the scope or content of the invention in any way.

### Example 1

### Glyceolin Bioavailabilty

Male ZDSD/Pco rats were bred onsite at PreClinOmics (PreCclinOomics, Indianapolis, Ind.), individually housed in suspended wire cages, and maintained on a 12:12 hour light-dark cycle under standard laboratory conditions with a controlled room temperature (20-21° C.). The protocol and all procedures were approved by the Institutional Animal Care and Use Committee of PreClinOmics. Rats with a 3 month bodyweight of approximately 500 g were chosen for the experiments since they would not yet have developed diabetes. Diabetic synchronization can be achieved by feeding a calorie dense diet. However, because a prediabetic model was needed for this study, the ZDSD/Pco rats received ground irradiated Purina 5008 chow (Ralston Purina, Belmont, Calif.) to maintain a prediabetic state throughout the study. Chow was placed in spill resistant jars for accurate food intake measurements and the rats had free access to drinking water.

Glyceollins were administered via oral gavage (3 mL) to rats in the fed state. The study design included the following groups (n=3 rats per group): vehicle (poloxamer 407; 7.5% in water), glyceollins dissolved in poloxamer to administer 30 mg/kg and 90 mg/kg. Blood levels of glyceollins were measured 0.5-, 1-, 2-, and 4-h after oral gavage. The animals were euthanized by decapitation and trunk blood was collected into EDTA coated tubes supplemented with aprotinin. Plasma was separated and stored at −80° C. until analysis by HPLC-ESI-MS/MS (FIG. 1). These data demonstrate that glyceollins are absorbed after oral administration into the circulation to some extent, exposing cells to the isoflavones. The plasma levels were low but sustained for 4 h.

### Example 2

### Oral Glucose Tolerance Test

Male ZDSD/Pco rats were bred onsite at PreClinOmics (PreCclinOomics, Indianapolis, Ind.), individually housed in suspended wire cages, and maintained on a 12:12 hour light-dark cycle under standard laboratory conditions with a controlled room temperature (20-21° C.). The protocol and all procedures were approved by the Institutional Animal Care and Use Committee of PreClinOmics. Rats with a 3 month bodyweight of approximately 500 g were chosen for the experiments since they would not yet have developed diabetes. Diabetic synchronization can be achieved by feeding a calorie dense diet. However, because a prediabetic model was needed for this study, the ZDSD/Pco rats received ground irradiated Purina 5008 chow (Ralston Purina, Belmont, Calif.) to maintain a prediabetic state throughout the study. Chow was placed in spill resistant jars for accurate food intake measurements and the rats had free access to drinking water.

Eight rats were randomly assigned to receive either glyceollins (30 mg/kg or 90 mg/kg) or vehicle at the onset of the photoperiod dark cycle as described in Example 1. An oral glucose tolerance test (OGTT) was performed as described below on dl of treatment.

After fasting for 5 h into the dark cycle of the photoperiod, glyceollins were administered via oral gavage as described above. There were 8 rats in each group for this experiment. On the 6^{th} h, the rats were dosed with glucose (2 g/kg, 10 ml/kg, p.o.). Tail vein blood was sampled for glucose measurement at −15-, 30-, 60-, 90-, and 120-minutes after the glucose challenge. Whole blood glucose levels were measured using an AlphaTrak blood glucose monitor (Abbott Laboratories, Abbott Park, Ill.).

The ZDSD/Pco rats were in a prediabetic state as evidenced by the fasting blood glucose value of 127.6±1.5 mg/dl (n=24). Blood glucose increased to a maximum level at 30 min after the oral glucose gavage, it remained elevated in the vehicle group until 60 min but was significantly (p<0.05) less at that time in both glyceollin groups (FIG. 2). Disposal of the circulating glucose during the 120 min period of the oral challenge was significantly (p<0.05) greater in both glyceollin groups than in the vehicle group (AUC for the OGTT was 26890±876, 24310±496, and 23401±754 mg/min/dl, for vehicle group (n=8), 30 mg/kg glyceollin group (n=8) and 90 mg/kg glyceollin group (n=8). There was no significant difference between the 2 glyceollin groups.

The data demonstrate that pretreatment with a mixture of 3 glyceollins improved the blood glucose response of prediabetic ZDSD rats to an oral glucose challenge.

### Example 3

### Activity in Adipocytes

To determine if one mechanism for glyceollins to improve the oral glucose challenge in a prediabetic model is by increasing the glucose uptake by fat, glyceollin pharmacology was studied in 3T3-L1 cells. Murine preadipocytes (Zen-Bio Inc.) were cultured using PM-1-L1 medium (Zen-Bio Inc.) containing Dulbecco's modified Eagle's medium (DMEM)/Ham's F-10 medium (1:1, v/v), HEPES 15 mM (pH 7.4), 10% (v/v) fetal bovine serum, penicillin (100 U/ml), streptomycin (100 mg/ml), and amphotericin B (0.25 μg/ml) in a humidified atmosphere (5% CO₂/95% air). After 3-4 days, confluent cells were placed in differentiation medium (DM-2-L1, Zen-Bio Inc.) containing DMEM/Ham's F-10 medium (1:1, v/v), HEPES 15 mM (pH 7.4), 3% (v/v) fetal bovine serum, biotin (33 μM), pantothenate (17 μM), human insulin (100 nM), dexamethasone (1 μM), penicillin (100 U/ml), streptomycin (100 μg/ml), amphotericin B (0.25 μg/ml), isobutylmethylxanthine (0.20 μM) and PPARγ agonist (10 μM) and further incubated in the humidified atmosphere for 3 days. The medium was then changed to AM-1-L1 medium (Zen-Bio Inc.) containing DMEM/Ham's F-10 medium (1:1, v/v), HEPES 15 mM (pH 7.4), 3% (v/v) fetal bovine serum, biotin (33 μM), pantothenate (17 μM), human insulin (100 nM), dexamethasone (1 μM), penicillin (100 U/ml), streptomycin (100 μg/ml), and amphotericin B (0.25 μg/ml). AM-1-L1 medium (i.e., adipocyte maintenance medium, such as those commercially provided by ZenBio©) was changed every 2-3 days during an additional 10 days of incubation.

Adipocytes were rinsed in sterile, fresh KRH buffer (HEPES pH=7.4, 1 mM CaCl₂, 1.2 mM MgSO₄, 1 mM KH₂PO₄, 1.4 mM KCl, 20 mM, 130 mM NaCl), and then preincubated for 24 h in KRH buffer. The buffer was removed, and adipocytes were incubated in KRH buffer containing glyceollins (at concentrations indicated, such as at 0.5 μM-5 μM) for the specified time period. 10 μL of [³H]-2-deoxy-D-glucose (Vitrax, Placentia, Calif.) diluted to 0.01 μCi/μL with D-glucose (100 mM) was added to each well and incubated 10 min in a 37° C. water bath. The supernatant was removed, and plates were rinsed rapidly three times with ice cold KRH. The final rinse was aspirated, taking care not to remove the cellular monolayer, then 500 μL ice cold RIPA buffer (Sigma-Aldrich, St. Louis, Mo.) was added to lyse the cells. The cellular content in each well was triturated with a 1 ml pipette several times to remove attached cells and cellular components from the bottom of the plate. Aliquots of 450 μL were transferred to vials containing 5 mL Ecolume scintillation fluid (MP Biomedical, Santa Ana, Calif.). The vials were mixed and counted for 10 min in an Applied Biosystems 1100 liquid scintillation counter using the factory preset window to detect tritium.

These adipocytes respond well to insulin stimulation (FIG. 3). Glucose was transported into the adipocytes in a dose-dependent manner (0.3 nM-300 nM insulin). Maximal stimulation was observed to be about 3-times that measured for basal glucose uptake and the concentration of insulin that produced half of that response (EC₅₀) was calculated to be 1.9±1.5 nM (n=6).

To determine whether the response of adipocytes to insulin is potentiated by glyceollins, 3T3-L1 differentiated cells were incubated with DMSO (vehicle control), 0.3 nM insulin, 5 μM glyceollins, or both glyceollin mix with insulin. Although the glucose uptake stimulated by insulin with glyceollins tended to be greater, the increase was not significantly different (FIG. 4). Surprisingly, the glyceollin blend was as efficacious as insulin in stimulating glucose uptake but less potent.

In order to study the dose—glucose uptake response to glyceollin, dose ranging studies were performed (FIG. 5). Glucose uptake was stimulated by 45 min exposure to glyceollins at doses ranging between 0.5 μM and 10 μM with an EC₅₀ of 2.40±0.43 μM and a maximal uptake of 2.04±0.24-fold stimulation above basal glucose uptake (n=3).

To determine the underlying mechanism for glyceollin stimulation of glucose uptake, the expression of the genes encoding GLUT1 and (GLUT4, which are the key glucose transporter protein genes expressed in adipocytes, was examined.

Adipocytes were grown in 6-well plates, as described above, and used at day 10-11 after initiation of differentiation. Adipocytes were rinsed in sterile KRH buffer, and then preincubated for 24 h in KRH buffer. The buffer was removed and adipocytes were treated with either DMSO as a vehicle, or glyceollins (at concentrations indicated, such as 1 μM or 10 μM) for 3 h. Total RNA was isolated using Trizol reagent (Invitrogen) and purified on RNeasy columns (Qiagen) according to the manufacturer's protocol. RNA quality and concentration was determined by absorbance at 260 nm and 280 nm. Total RNA was reverse-transcribed using a QUANTITECT Reverse Transcription kit (Qiagen). The sequences of the forward primer, reverse primer, and TaqMan probes for GLUT1, GLUT4, and the housekeeping gene ribosomal protein L32 (RPL32) (NM_{—}172086) are described, in Obesity, 2008, 16:1208-1218. The reactions were performed in 96-well plates in a CFX96 Real-Time PCR Detection Systems (Bio-Rad). The thermal cycle conditions were as follows: 2 min at 50 C and 10 min at 95 C, followed by 50 cycles at 95 C for 15 s each and 60° C. for 60 s. The ΔΔC_{T} method of relative quantification was used to determine the fold change in expression. This was done by first normalizing the resulting threshold cycle (C_{T}) values of the target mRNAs to the C_{T} values of the internal control Rp132 in the same samples. Those data were compared to the DMSO control.

Expression of both GLUT1 and GLUT4 were significantly increased in cells exposed to glyceollin ranging from 1 μM-10 μM (FIG. 6).

These findings establish that a mechanism for glyceollin-mediated glucose uptake into fat cells is by increasing the expression of both GLUT1 and GLUT4 genes. GLUT1 is thought to be responsible for basal glucose uptake by adipocytes and most other cells. GLUT4 is also expressed by adipose and other insulin target tissues. It is thought to be responsible for insulin-stimulated glucose uptake. Both GLUTs are expressed by 3T3-L1 cells after they differentiate into mature adipocytes. Thus, that glyceollins may act in concert with insulin or independently of the hormone to stimulate glucose uptake by adipocytes.

### Example 4

### Chronic Administration of Glyceollin to Prediabetic Rats

A blend of the 3 glyceollins (glyceollin I, glyceollin II and glyceollin III) to prediabetic ZDSD/Pco rats improves the blood glucose response to an oral glucose challenge (see Example 2). It is also demonstrated that glyceollin is only partially bioavailable after oral administration since plasma levels during 3 h after administration of either 30 mg/kg or 90 mg/kg were low (see Example 1). A study was performed using ZDSD/Pco rats to determine if oral administration of the glyceollins alters the GI microbiome and body composition.

The rats were treated with oral doses of the glyceollin blend (90 mg/kg) for 11d and the microbiota taxa in feces was analyzed before treatment and on day 11 of treatment. A Beckman Synchron CX4 random-access multianalyzer (Beckman Coulter, Inc., Brea, Calif.) was used to measure glucose (cat #OSR6121), cholesterol (cat #OSR6116) and triglycerides (cat #OSR6018) in plasma at the terminal bleed. Active GLP-1 and insulin were measured using a Meso Scale Discovery multiplex instrument and the K11159c-1 kit (Meso Scale Discovery, Inc., Gaithersburg, Md.). Leptin was measured by ELISA using an ALPCO Immunoassay rat/mouse leptin kit (22-LEPMS-E01).

Differences in abundance of 3 genera were observed when comparing baseline to the microbiota signature of feces obtained on day 11 of glyceollin treatment (Table 1). In particular, there was a dramatic bloom observed in species of Blautia. No shift in diversity or abundance was observed in taxa from vehicle treated rats when comparing fecal microbiota at pretreatment with that from treatment d11.

TABLE 1
Significant changes in 3 genera of microbiota after
only 11 days of treatment with a glyceollin blend.

| | Pretreatment | Day 11 | |
|---|---|---|---|
| | Abundance¹ | Abundance | |
| | (% of total, | (% of total, | |
| Genus | mean ± SEM) | mean ± SEM) | Probability |
| Blautia | 9.70 ± 0.56 | 20.68 ± 2.21 | 0.0005 |
| Peptostreptococcaceae | 0.00 ± 0.00 | 3.60 ± 1.34 | 0.01 |
| Ruminococcaceae | 3.87 ± 0.58 | 1.91 ± 0.54 | 0.02 |

¹Analytical data are from MultiTag Sequencing that was performed by Metabiomics (http://metabiomics.com/services/).

This change in the fecal profile of microbiota was significantly correlated (p<0.05) to a decrease in body weight (from 529.25±8.15 g to 529.00±10.32 g, vehicle group; from 529.88±5.84 to 523.88±6.44 g, glyceollin blend) that was primarily a consequence of decreased fat mass (FIG. 7).

The shift in fecal microbiota profile was also significantly correlated (p<0.05) to decreases in plasma leptin as well as increases in plasma GLP-1 (FIG. 8) and plasma insulin (FIG. 9).

The glyceollin blend stimulated a dramatic bloom in species of Blautia after just 11d of treatment. Species of this genus are hydrogen-consuming organisms that also have genes indicating that they can process polyphenolic molecules and can synthesize acetate (Int. J. Syst. Evol. Microbiol., 2008, 58:1896-902). Dietary components such as fiber that reach the colon are fermented principally to SCFAs, but hydrogen and carbon dioxide are also generated in that process. Microbial disposal of the hydrogen generated during anaerobic fermentation in the human colon is important for optimal functioning of this ecosystem (for review see Annu. Rev. Food Sci. Technol., 2010, 1:363-95). There are 2 other major groups of hydrogen-consuming microorganisms found in the colon, the methanogens and sulfate reducing bacteria. Both appear to occur mainly for hydrogen utilization and are in competition with each other as well as with the species of Blautia.

In a simplified model of human gut community relationships, transplantation of germ-free mice with Bacteroides thetaiotaomicron (capable of fermenting carbohydrate to SCFAs and hydrogen) and Methanobrevibacter smithii (capable of utilizing hydrogen and carbon to produce methane) but not a colonization of B. thetaiotaomicron with Desulfovibrio piger (capable of reacting hydrogen with sulfur to produce hydrogen sulfide), resulted in increased serum acetate levels, increased liver triglycerides, and increased adiposity (Proc Natl Acad Sci. USA, 2006, 103:10011-10016). Transplanting Bacteroides thetaioaomicron with Blautia hydrogenotrophica (capable of reacting hydrogen with carbons from fermentation to produce acetate) results in much greater circulating acetate levels than cotransplantation of B. thetaiotaomicron with M. smithii (J Biol Chem, 2010, 285: 22082-22090). Unfortunately, resulting liver triglycerides and mass of fat depots were not reported in that colonization pairing study. However, these data suggest that one type of hydrogen utilizing bacteria is more conducive for the host to accrue calories and the others are best at producing either acetate or hydrogen sulfide (often associated with colon pathology) when coupled with a carbohydrate fermenting bacteria. Without wishing to be bound by theory, it is believed herein that glyceollin-stimulated bloom in Blautia creates competition among the other 2 classes of hydrogen-consuming microbiota in the gut for hydrogen. Consequently, greater acetate levels in the colon that serve as ligands for satiety hormones as well as serve to generate an inactive ghrelin will induce decreased adipocity. Indeed, like hydrogen, the glyceollins are strong reducing agents and unlike the methanogens or sulfate reducing bacteria, Blautia are capable of processing molecules like polyphenolics (J Biol Chem, 2010, 285: 22082-22090).

### Example 5

### Human Study Utilizing Activated Soy Pod Fiber to Correct the GI Dysbiosis Observed in Type 2 Diabetes, Improve Glucose Regulation and Improve Body Composition

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This is designed to exemplify that overweight subjects with impaired fasting blood glucose on an ad libitum diet who take Activated Soy Pod Fiber either within 1 hour prior to meal 1 or within 1 hour prior to meal 2, as well as within 1 hour prior to meal 3 for 4 weeks, will:

1. Eliminate stool with an increased small chain fatty acids, decreased methane and hydrogen sulfide gases, increased acetate and increased antioxidants when compared to stool analyzed at the start of the intervention, and when compared to subjects consuming a placebo, and
2. Have an improved oral glucose tolerance test (OGTT) as measured by blood glucose and insulin levels before, during, and at 120 minutes after ingestion of 75 g glucose when compared to their initial OGTT, and when compared to subjects consuming placebo, and
3. Have lower overnight fasting blood glucose levels as measured by a blood glucose monitor before ingesting a morning meal when compared to their overnight fasting blood glucose values at the start of the intervention, and when compared to subjects consuming placebo.
4. Experience an improved body composition as measured by a decrease in body weight, a decrease in body fat or % body fat, a decrease in waist circumference measurements, and
5. Experience decreased appetite before a meal, increased satiety during the meal, when compared to subjects consuming placebo, and
6. Will be found to have elevated GLP-1 as well as PYY levels with reduced active ghrelin levels after a standardized meal when the values are compared to those of subjects consuming placebo on week 3 of the intervention.
In this study, subjects consume either 180 ml of Activated Soy Pod Fiber formula or a placebo containing the same total dietary fiber level as Activated Soy Pod Fiber but as inactive cellulose orally within 1 hour prior to consumption of either meal 1 or meal 2 and within 1 hour prior to consumption of meal 3 each day. Placebo formula contains cellulose with food coloring and flavor to match the total dietary fiber content of Activated Soy Pod Fiber. Placebo is prepared by Merlin Development at the same time they prepare Activated Soy Pod Fiber in a palatable easy to mix powder.

Subjects report weekly for measurements and assessment of any side effects. They are asked to collect a stool sample before initiating either Activated Soy Pod Fiber or placebo intervention as well as at the end of the 4 week treatment period. They are also asked to record any side effects and their frequency (checklist assessment). They are asked to record appetite (how hungry are you) and satiety (how full are you) during the standardized meal at the 3^{rd} week of intervention. They are provided with the proper paper work to record these.

### Subject Screening and Selection

A total of 30 subjects is selected, 15 assigned to Activated Soy Pod Fiber and 15 assigned to placebo.

### Qualifications of Subjects

1) Healthy men and women between the ages of 18 and 70 with a BMI between 25 and 45 are eligible. 2) Fasting blood glucose between 100 and 200 mg/dl. 3) Stable weight over 2 months

Subjects Excluded from Study

### People who:

a) take medications affecting glucose,
b) take medications affecting insulin,
c) take medications affecting body weight,
d) take medications affecting bacterial flora,
e) have intestinal disease or a recent history of intestinal disease,
f) have had surgery on stomach or intestine,
g) are hypothyroid,
h) are pregnant,
i) have heart disease.

### Laboratory Evaluation

Different tests are performed at the screening of potential participants, at the beginning of the study, and at the end of the 4 week treatment period.

1) Screening: Subjects are screened to exclude hypothyroidism, pregnancy, and heart disease. The following tests can suffice for this: T4 (thyroxin), T3 (triiodotyronine), TSH (thyroid stimulating hormone), urine pregnancy test, blood pressure & ECG (electrocardiogram).
2) Beginning of study: Subjects passing the initial screen are evaluated at the beginning of Week #1 as follows:
a) Fasting blood glucose and insulin levels
b) SMA 20 (Sequential multi-channel analysis with computer-20, a metabolic panel with 20 different analytes), including, uric acid, and liver function tests
c) Triglycerides
d) Cholesterol, including fractions
e) Glycosylated hemoglobin A1 (HgbA1)
f) Weight, taken on the same scale each time
g) Body fat % and total body fat, determined by DXA (dual-energy X-ray absorptiometry).
h) Height
i) Waist and hip measurements
j) Blood glucose, insulin, GLP-1, PYY and ghrelin responses to a 75 g oral glucose challenge
k) Assessment of appetite and satiety using a visual analog scale
l) Stool is collected and stored frozen but not analyzed until the end of study.
   3) End of study assessment:
a) All labs and assessments done in step 2 at beginning of study,
b) Analysis of the fecal microbiome DNA from both the initial sample and the final sample.
c) Analysis of feces that includes: pH, SCFAs, lactoferrin, white blood cells, mucus, secreted immunoglobulin A, anti-gliadin secreted immunoglobulin A, pathogenic bacteria, yeast, fungi, parasites, triglycerides, branched chain fatty acids, long chain fatty acids, and cholesterol.

### Study Design

Subjects selected for participation are allowed an ad libitum diet and are given an evaluation sheet to assess their appetite and satiety before and after a meal. Foods excluded include alcohol. Low calorie liquids are stressed in place of high calorie liquids such as fruit juices, milk, sweet tea (tea with sugar), regular soft drinks, coffee with sugar, etc. The subjects are randomly assigned to either Activated Soy Pod Fiber or placebo treatments. Both the experimenter and the subjects are blinded to who receive Activated Soy Pod Fiber or the placebo. The subjects are encouraged to consume either treatment within 1 hour prior to either breakfast or lunch and within 1 hour prior to dinner.

### Duration

Subjects are given a 4 weeks supply of either Activated Soy Pod Fiber or placebo at the onset and are instructed to consume the entire 180 ml volume within 1 hour prior to either meals 1 or 2, as well as another 180 ml volume containing either Activated Soy Pod Fiber or placebo within 1 hour prior to meal 3. Ad libitum diets are followed for 4 weeks, but the volunteers are instructed to consume either Activated Soy Pod Fiber or placebo as their only between meal snack.

### Outcome

This study is designed to exemplify that Activated Soy Pod Fiber:

1) Eliminate stool with increased small chain fatty acids, decreased methane and hydrogen sulfide gases, increased acetate and increased antioxidants when compared to stool analyzed at the start of the intervention, and when compared to subjects consuming a placebo
2) Improves the blood glucose and insulin responses to an OGTT by decreasing the areas under the insulin curve (improved insulin sensitivity);
3) Decrease fasting blood glucose values
4) Produces weight loss, loss of body fat, and (or) decrease of body fat %
5) Increases GLP-1 and PYY response to the oral glucose challenge and decreases the fasting ghrelin levels at 1 hour after the both the OGTT and the standardized meal when comparing final values to the initial measurements of the OCGTT, and when comparing to those findings of the placebo group during the standardized meal; and
6) Decreases stool pH;
7) Increases stool SCFA.
8) Increased stool lactoferrin
9) Decreased stool white blood cells
10) Decreased stool mucus
11) Increased stool secreted immunoglobulin A
12) Increased stool anti-gliadin secreted immunoglobulin A
13) Decreased stool pathogenic bacteria
14) Decreased stool yeast, fungi, and parasites
15) Increased stool triglycerides
16) Decreased stool branched chain fatty acids
17) No change in stool long chain fatty acids
18) Increased stool cholesterol.
If subjects took Activated Soy Pod Fiber for periods longer than 4 weeks, particularly for at least 8 weeks, the subjects would experience significant weight loss that was primarily fat loss.

### Example 6

### Human Study Utilizing Activated Soy Pod Fiber in Combination with an Inhibitor of Dipeptidyl Peptidase-4 (DPP-4) to Correct the GI Dysbiosis Observed in Type 2 Diabetes to and to Improve Glucose Regulation by Sustained Elevation of GLP-1

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This study is designed to exemplify that type 2 diabetic (T2D) subjects with insulin resistance on an ad libitum diet who take a DPP-4 inhibitor and Activated Soy Pod Fiber within 1 hour prior to either meal 1 or meal 2, as well as within 1 hour prior to meal 3 for 4 weeks:

1. Eliminate stool characterized as normal at termination of treatment when compared to initiation of treatment and when compared to those only taking the DPP-4 inhibitor, and
2. Have improved insulin sensitivity when compared to both initiation of the study and when compared to those only taking a DPP-4 inhibitor. Insulin sensitivity is measured by an oral glucose tolerance test (OGTT). This is performed by measuring blood glucose and insulin levels before, during, and at 120 minutes after ingestion of 75 g glucose when compared to their initial OGTT, and
3. Have improved fasting blood glucose values when compared to those only taking the DPP-4 inhibitor, and
4. experience an improved body composition as measured by a decrease in body weight, a decrease in body fat or % body fat, a decrease in waist circumference measurements when compared to their baseline values and when compared to those only taking the DPP-4 inhibitor, and
5. experience decreased appetite before a standardized meal, increased satiety during that meal when compared to their baseline values and when compared to those only taking the DPP-4 inhibitor, and
6. are found to have elevated GLP-1 as well as PYY levels with reduced ghrelin levels at 1 hour after the both the OGTT and the standardized meal when the values are compared to their baseline values and when compared to those only taking the DPP-4 inhibitor.

### General

In this study, T2D patients are randomly assigned to either consume 180 ml of Activated Soy Pod Fiber or a placebo formula containing cellulose orally within 1 hour prior to either meals 1 or 2 as well as within 1 hour prior to meal 3 each day. Patients and experimenters are blinded to this assignment. All patients are also instructed to take sitagliptin (Januvia®) at the recommended dose of 100 mg, once per day in the morning prior to meal 1 as a treatment to manage their diabetes.

Subjects report weekly for measurements and assessment of any side effects. They are asked to collect a stool sample before the initiation of the trial as well as at the end of the 4 week treatment period. They are also asked to record any side effects and their frequency (checklist assessment). They are asked to record appetite (how hungry are you) and satiety (how full are you) during the OGTT at both the onset and at the end of the trial as well as before and during a standardized meal at the 3^{rd} week of treatments. They are provided with the proper paper work to record these.

### Subject Screening and Selection

A total of 24 subjects is selected. 12 will be randomly assigned to receive sitagliptin+placebo (a cellulose solution that contains the same total dietary fiber content as Activated Soy Pod Fiber and mimics Activated Soy Pod Fiber in color and taste) or sitagliptin+Activated Soy Pod Fiber.

### Qualifications of Subjects

1) T2D men and women between the ages of 18 and 70 with a BMI between 25 and 45 are eligible.
2) Fasting blood glucose between greater than 125 mg/dl.
3) Stable weight over 2 months
Subjects Excluded from the Study

### People who:

a) take medications affecting glucose other than sitagliptin,
b) take medications affecting insulin other than sitagliptin,
c) take medications affecting body weight,
d) take medications affecting bacterial flora,
e) have intestinal disease or a recent history of intestinal disease,
f) have had surgery on stomach or intestine,
g) are hypothyroid,
h) are pregnant,
i) have heart disease.

### Laboratory Evaluation

Different tests are performed at the screening of potential participants, at the beginning of the study, and at the end of the 4 week treatment period.

4) Screening: Subjects are screened to exclude hypothyroidism, pregnancy, and heart disease. The following tests can suffice for this: T4, T3, TSH, urine pregnancy test, blood pressure & ECG. Fasting blood glucose, fasting insulin and HgbA1 levels are also measured as an assessment of their diabetic state.
5) Beginning of study: Subjects passing the initial screen are evaluated at the beginning of Week #1 as follows:
a) Fasting blood glucose, insulin, and HgbA1 levels.
b) SMA 20, including uric acid and liver function tests

### c) Blood Triglycerides

d) Plasma Cholesterol, including fractions
f) Weight, taken on the same scale each time
g) Body fat % and total body fat, determined by DXA.

### h) Height

i) Waist and hip measurements
j) Blood glucose, insulin, GLP-1, PYY and ghrelin responses to a 75 g oral glucose challenge
k) Assessment of appetite and satiety before, during and after a standardized meal using a visual analog scale
l) Stool is collected into a preservative and analyzed within 1 week at baseline and at the end of study.
6) End of study assessment:
c) all labs and assessments done in step 2 at beginning of study,
d) Analysis of the fecal microbiome DNA from both the initial sample and the final sample.
e) stool pH;
f) stool SCFA.
g) stool lactoferrin
h) stool white blood cells
i) stool mucus
j) stool secreted immunoglobulin A
k) stool anti-gliadin secreted immunoglobulin A
l) stool pathogenic bacteria
m) stool yeast, fungi, and parasites
n) stool triglycerides
o) stool branched chain fatty acids
p) stool long chain fatty acids
q) stool cholesterol.

### Study Design

Patients selected for participation are allowed an ad libitum diet and are given an evaluation sheet to assess their appetite and satiety. Foods excluded include alcohol. Low calorie liquids are stressed in place of high calorie liquids such as fruit juices, milk, sweet tea (tea with sugar), regular soft drinks, coffee with sugar, etc. All 24 patients are also instructed to take sitagliptin (Januvia®) at the recommended dose of 100 mg, once per day in the morning with or without food as a treatment to manage their diabetes. 12 patients are randomly selected to also consume Activated Soy Pod Fiber before 2 of 3 daily meals and the remaining 12 patients are instructed to consume a placebo before 2 of 3 daily meals. Patients and investigators are blinded to whether the snack replacement is placebo or Activated Soy Pod Fiber.

### Duration

Subjects are given a 4 weeks supply of sitagliptin and either Activated Soy Pod Fiber or placebo at the onset and are instructed to consume the entire 180 ml volume of either snack replacement within 1 hour prior to either meals 1 or 2, as well as another 180 ml volume of snack replacement within 1 hour prior to meal 3. All subjects are required to take 1 tablet of sitagliptin daily (100 mg) in the morning with or without food. Ad libitum diets are followed for 4 weeks.

### Outcome

This study is designed to exemplify that Activated Soy Pod Fiber:

1) Shifts bacterial species in fecal samples from those documented in feces from T2D to those typical of non-diabetic subjects when samples at the end of study from those assigned to Activated Soy Pod Fiber are compared to samples at the onset of study and when subjects taking sitagliptin+Activated Soy Pod Fiber are compared to patients taking sitagliptin+placebo.
2) Improves the blood glucose and insulin responses to an OGTT by decreasing the areas under the insulin curve (improved insulin sensitivity) when subjects taking sitagliptin+Activated Soy Pod Fiber are compared to patients taking sitagliptin+placebo
3) produces weight loss, loss of body fat, and (or) decrease of body fat % when patients assigned to Activated Soy Pod Fiber are compared to samples at the onset of study and when subjects taking sitagliptin+Activated Soy Pod Fiber are compared to patients taking sitagliptin+placebo.
4) produces decreased fasting blood glucose levels when subjects taking sitagliptin+Activated Soy Pod Fiber are compared to patients taking sitagliptin+placebo, and
5) Increases GLP-1 and PYY response to the oral glucose challenge and decreases the fasting ghrelin levels prior to the OGTT when patients assigned to Activated Soy Pod Fiber are compared to samples at the onset of study and when subjects taking sitagliptin+Activated Soy Pod Fiber are compared to patients taking sitagliptin+placebo,
6) Patients assigned to the Activated Soy Pod Fiber arm when compared to samples at the onset of study and subjects taking sitagliptin Activated Soy Pod Fiber when compared to patients taking sitagliptin+placebo are expected to have the following changes in stool samples:
a) Decreases stool pH;
b) Increases stool SCFA.
c) Increased stool lactoferrin
d) Decreased stool white blood cells
e) Decreased stool mucus
f) Increased stool secreted immunoglobulin A
g) Increased stool anti-gliadin secreted immunoglobulin A
h) Decreased stool pathogenic bacteria
i) Decreased stool yeast, fungi, and parasites
j) Increased stool triglycerides
k) Decreased stool branched chain fatty acids
l) No change in stool long chain fatty acids
m) Increased stool cholesterol.
If subjects took Activated Soy Pod Fiber with other DPP-IV inhibitors or other formulations of sitagliptin, the subjects are expected to also have significantly improved glucose regulation.

### Example 7

### Study Utilizing Activated Soy Pod Fiber Snack Replacement to Increase the Ratio of Gastrointestinal Microbiota in Phylum Bacteriodetes to Correct the GI Dysbiosis Observed in Overweight and Obese Children, and Improve Glucose Gegulation and Improve Body Composition

### Subjects and Methods

The required number of children are properly screened to fulfill the necessary qualifications and their parental consent is obtained, appropriate laboratory evaluations are performed, measures of positive primary and secondary outcome responses are recorded, adverse events are documented, and children and their parents are adequately followed-up.

### Overview

This study is designed to exemplify that overweight children with prediabetes or at high risk of developing T2D (type 2 diabetes) on an ad libitum diet who take Formula A (identical active ingredients to Activated Soy Pod Fiber but formulated in a child friendly delivery system such as ice cream, jelled animals, cookies, etc.) within 1 hour prior to either meal 1 or meal 2, as well as within 1 hour prior to meal 3 for 4 weeks:

1. Eliminate stool characterized as normal diversity when compared to the start of the intervention, and
2. Have an improved oral glucose tolerance test (OGTT) as measured by blood glucose and insulin levels before, during, and at 120 minutes after ingestion of 1.75 g glucose/kg body weight upto 75 g glucose when compared to their initial OGTT, and
   3. experience an improved body composition as measured by a decrease in body weight, a decrease in body fat or % body fat, a decrease in waist circumference measurements, and
   4. Experience decreased fasting blood glucose levels
   5. experience decreased appetite before a meal, increased satiety during the meal, and
   6. are found to have elevated GLP-I as well as PYY levels with reduced ghrelin levels 1 hour after the OGTT when the values are compared to those at the initiation of the trial.

### General

In this study, children consume 6 jelled animals of Formula B formula (each jelled animal contains about 20 g of Formula B) within 1 hour prior to either meals 1 or 2 as well as within 1 hour prior to meal 3 each day.

Subjects report weekly for measurements and assessment of any side effects. They are asked to collect a stool sample before initiating Formula B intervention as well as at the end of the 4 week treatment period. They are also asked to report side effects to their parents who record them and their frequency (checklist assessment). The parents are instructed to ask and to record appetite (how hungry are you) and satiety (how full are you) before, during, and after a standardized 3^{rd} meal at the beginning of study and at the end. The investigators score the same assessment during the OGTT at both the onset and at the end of the trial as well as at home. The parents are provided with the proper paper work to record these.

### Subject Screening and Selection

A total of 10 children is selected.

### Qualifications Of Subjects

1) Healthy prepubertal boys and girls between the ages of 7 and 12 with a BMI between 25 and 30 are eligible. 2) Fasting blood glucose between 100 and 125 mg/dl.

### Subjects Excluded Form the Study

### Children who:

a) take medications affecting glucose,
b) take medications affecting insulin,
c) take medications affecting body weight,
d) take medications affecting bacterial flora,
e) have intestinal disease or a recent history of intestinal disease,
f) have had surgery on stomach or intestine,
g) are hypothyroid.

### Laboratory Evaluation

Different tests are performed at the screening of potential participants, at the beginning of the study, and at the end of the 4 week treatment period.

### Screening

Children are screened to exclude hypothyroidism and puberty. The following tests can suffice for this: T4, T3, TSH, a physical exam, and in questionable cases based on the physical exam or peripubertal presentations, a gonadotropin-releasing hormone challenge test.

### Beginning of Study

Children passing the initial screen are evaluated at the beginning of WEEK #1 as follows:

a) Fasting blood glucose and insulin levels.
b) SMA 20, including uric acid and liver function tests

### c) Triglycerides

d) Cholesterol, including fractions
e) Glycosylated hemoglobin A1 (HgbA1)
f) Weight, taken on the same scale each time
g) Body fat % and total body fat, determined by DXA.

### h) Height

i) Waist and hip measurements
j) Blood glucose, insulin, GLP-I, PYY and ghrelin responses to a 1.75 g/kg (up to 75 g) oral glucose challenge
k) Assessment of appetite and satiety using a visual analog scale
l) Stool is collected in a preservative and analyzed within 1 week.

### End of Study Assessment

a) all labs and assessments done in step 2 at beginning of study,
b) Analysis of the fecal microbiome DNA from both the initial sample and the final sample.

### Study Design

Children selected for participation are allowed an ad libitum diet and their parents are given an evaluation sheet to assess their appetite and satiety. Low calorie liquids are stressed in place of high calorie liquids such as fruit juices, milk, regular soft drinks, coffee with sugar, etc. The children are encouraged to consume Formula B as their only between meal snack. Other snacks such as candy, ice cream, milk shakes, cookies, potato chips, etc. are discouraged.

### Duration

Children are given a 4 weeks supply of Formula B at the onset and are instructed to consume the entire 6 jelled animals containing Formula B within 1 hour prior to either meal 1 or 2, as well as another 6 jelled animals containing Formula B within 1 hour prior to meal 3. Ad libitum diets are followed for 4 weeks, but the children are instructed to consume Formula B as their only between meal snack.

### Outcome

This study is designed to exemplify that Formula B:

1) Shifts bacterial species in fecal samples from those documented for obese children to those typical of healthy lean children and adolescents when samples at the end of study are compared to samples at the onset of study.
2) Improves the blood glucose and insulin responses to an OGTT by decreasing the areas under the insulin curve (improved insulin sensitivity)
3) produces weight loss, loss of body fat, and (or) decrease of body fat %
4) Increases GLP-1 and PYY response to the oral glucose challenge and decreases the fasting ghrelin levels prior to the OGTT when comparing final values to the initial measurements.
5) Alters the following stool characteristics:
a) Decreases stool pH;
b) Increases stool SCFA.
c) Increased stool lactoferrin
d) Decreased stool white blood cells
e) Decreased stool mucus
f) Increased stool secreted immunoglobulin A
g) Increased stool anti-gliadin secreted immunoglobulin A
h) Decreased stool pathogenic bacteria
i) Decreased stool yeast, fungi, and parasites
j) Increased stool triglycerides
k) Decreased stool branched chain fatty acids
l) No change in stool long chain fatty acids
m) Increased stool cholesterol.

If children took Activated Soy Pod Fiber for periods longer than 4 weeks as a snack replacement, particularly for at least 8 weeks, the children would experience significant weight loss that was primarily fat loss.

### Example 8

### Human Study Utilizing Either Activated Soy Pod Fiber or a Placebo to Shift the Gastrointestinal Microbiota in Irritable Bowel Syndrome (IBS) to that Characterized in Healthy Individuals

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This randomized, placebo-controlled clinical trial is designed to exemplify the efficacy and tolerability of Activated Soy Pod Fiber in diarrhea-predominant humans with IBS. Subjects assigned to consume Activated Soy Pod Fiber but not a placebo, within 1 hour prior to consuming either meal 1 or meal 2, as well as within 1 hour prior to consuming meal 3 for 4 weeks:

1. Eliminate firm stool containing a normal diversity of GI microbiota within 1 week of starting treatment
2. Report adequate relief for all 4 weeks, and
3. Report decreased urgency, and
4. Report decreased stool frequency, and

### General

In this study, subjects are randomly selected to consume 180 ml of either Activated Soy Pod Fiber formula or placebo formula orally within 1 hour prior to ingestion of either meals 1 or 2 as well as within 1 hour prior to consumption of meal 3 each day. The subjects and experimenters are blinded to the treatment assignments. Placebo formula contains cellulose with food coloring and flavor to match the total dietary fiber content of Activated Soy Pod Fiber. Placebo is prepared by Merlin Development at the same time they prepare Activated Soy Pod Fiber. Both formulations are coded by Merlin Development and the code is maintained with them as well as is held in confidence by a pharmacist at the study clinic until all data are collected at the end of study.

Subjects report weekly for measurements and assessment of any side effects. They are asked to collect a stool sample before initiating Activated Soy Pod Fiber or Placebo intervention as well as at the end of the 4 week treatment period. During the screening, treatment, and follow-up periods, daily and weekly symptom data are collected using an interactive telephone-based system.

Pain and bowel function data are collected during the screening phase to ensure that patients had a suitable symptom level at study entry. Severity of pain and discomfort was assessed daily on a 5-point scale (0, none; 1, mild; 2, moderate; 3, intense; and 4, severe). Stool consistency data are monitored daily and scored as follows: 1, very hard; 2, hard; 3, formed; 4, loose; and 5, watery. Absence of stool was assigned a value of 0. Patients also record their IBS symptoms urgency (0%, feel no need to evacuate—100%, feel severe need to evacuate), stool frequency (# of stools per day), bloating (0, no sensation of extended abdomen; 1, mild; 2, moderate; 3, severe) and sense of incomplete evacuation (0, sensation of complete evacuation; 1, incomplete; 2, constipated) daily during the treatment and follow-up phases.

### Subject Screening and Selection

Patients with IBS and a diarrhea-predominant bowel pattern aged 18 years or older are enrolled in this study if their symptoms fulfilled the Rome I criteria for IBS for at least 6 months. Patients undergo a 2-week screening evaluation to confirm sufficient level of pain and stool consistency before randomization. Since no objective criteria exist for subgrouping of IBS patients, physicians are asked to assess patients according to predominant pattern of bowel function based on the patient's disease history. Physicians are provided with a guideline based on the percentage of time the patient had experienced diarrhea. If diarrhea is present for ≧75% of the time, then the patient is classified as being diarrhea predominant.

Patients are excluded if they are pregnant, breastfeeding, or not using approved methods of contraception (if of child-bearing potential); if an unstable medical or other gastrointestinal condition exists; if there is a major psychiatric disorder or substance abuse within the previous 2 years; if an investigational drug was used within 30 days of the screening phase; or if a prohibited concurrent medication (likely to interfere with gastrointestinal tract function or analgesia) was used within 7 days before entering the screening phase. Pain and bowel function data are collected during the screening phase to ensure that patients had a suitable symptom level at study entry as described above.

### Symptom and Laboratory Evaluation

Evaluations are performed at the screening of potential participants, at the beginning of the study, daily, and at the end of the 4 week treatment period.

1) Beginning of study and daily
a) Severity of pain and discomfort is assessed on a 5-point scale (0, none; 1, mild; 2, moderate; 3, intense; and 4, severe).
b) Stool consistency data are scored as follows: 1, very hard; 2, hard; 3, formed; 4, loose; and 5, watery. Absence of stool was assigned a value of 0.
c) Urgency (0%, feel no need to evacuate—100%, feel severe need to evacuate),
d) Stool frequency (# of stools per day)
e) Bloating (0, no sensation of extended abdomen; 1, mild; 2, moderate; 3, severe)
f) Sense of incomplete evacuation (0, sensation of complete evacuation; 1, incomplete; 2, constipated)
g) Body weight.
2) Beginning of study
Stool is collected and stored frozen but not analyzed until the end of study.
3) End of study assessment
Analysis of the fecal microbiome DNA and complete stool analysis from both the initial sample and the final sample.

### Study Design

Subjects selected for participation are allowed an ad libitum diet. Foods excluded include alcohol. The subjects are encouraged to consume either Activated Soy Pod Fiber or Placebo within 1 hour prior to 2 meals each day with ingestion of the test agent being mandatory prior to the 3^{rd} meal.

Subjects are given a 4 week supply of ether Activated Soy Pod Fiber or Placebo at the onset and are instructed to consume the entire 180 ml volume containing either formula within 1 hour prior to either meals 1 and 2, as well as another 180 ml volume containing either formula within 1 hour prior to meal 3.

Subjects report weekly for measurements and assessment of IBS symptoms. During the screening and treatment periods, daily symptom data are collected using an interactive telephone-based system.

### Outcome

This study is designed to exemplify that Activated Soy Pod Fiber and not Placebo:

1) Improves severity of pain and discomfort;
2) Increases stool consistency;
3) Decreases urgency to evacuate,
4) Decreases stool frequency;
5) Decreases bloating
6) Increases sense of complete evacuation.
7) Shifts stool profile to one representative of a healthy GI microbiome
a) Decreases stool pH;
b) Increases stool SCFA.
c) Increased stool lactoferrin
d) Decreased stool white blood cells
e) Decreased stool mucus
f) Increased stool secreted immunoglobulin A
g) Increased stool anti-gliadin secreted immunoglobulin A
h) Decreased stool pathogenic bacteria
i) Decreased stool yeast, fungi, and parasites
j) Increased stool triglycerides
k) Decreased stool branched chain fatty acids
l) No change in stool long chain fatty acids
m) Increased stool cholesterol.

Utilization of Activated Soy Pod Fiber to treat idiopathic diarrhea such as a parasitic infection, a viral infection and a symptomatic response to a food is expected to also improve the severity of pain and discomfort, increase stool consistency, decrease the urgency to evacuate, decrease stool frequency, and decrease the sensation of bloating.

### Example 9

### Human Study Utilizing Either Activated Soy Pod Fiber or a Placebo to Treat Gestational Diabetes

### Subjects and Methods

The required number of subjects are properly screened to fulfill the necessary qualifications, appropriate laboratory evaluations are performed, measures of positive primary and secondary outcome responses are recorded, adverse events are documented, and patients are adequately followed-up.

### Overview

This randomized, placebo-controlled clinical trial is designed to exemplify the efficacy and tolerability of Activated Soy Pod Fiber in gestational diabetes. Subjects assigned to consume Activated Soy Pod Fiber but not a placebo, within 1 hour prior to consuming either meal 1 or meal 2, as well as within 1 hour prior to consuming meal 3 for 4 weeks:

1. Have improved glycemic control
2. Have acceptability of the treatment

### General

In this study, subjects are randomly selected to consume 180 ml of either Activated Soy Pod Fiber formula or placebo formula orally within 1 hour prior to ingestion of either meals 1 or 2 as well as within 1 hour prior to consumption of meal 3 each day. The subjects and experimenters are blinded to the treatment assignments. Placebo formula contains cellulose with food coloring and flavor to match the total dietary fiber content of Activated Soy Pod Fiber. Placebo is prepared by Merlin Development at the same time they prepare Activated Soy Pod Fiber. Both formulations are coded by Merlin Development and the code is maintained with them as well as is held in confidence by a pharmacist at the study clinic until all data are collected at the end of study.

Subjects report weekly for measurements and assessment of any side effects. They are asked to collect a stool sample before initiating Activated Soy Pod Fiber or Placebo intervention as well as at the end of the 4 week treatment period. During the screening, treatment, and follow-up periods, daily blood glucose data are collected by the patient using finger stick.

### Subject Screening and Selection

Women who are at least 18 years of age with gestational diabetes at 24-28 weeks (American Diabetes Association (ADA) criteria), who need an intervention treatment following the failure of the diet and exercise, and who are not known type 1 or type 2 diabetics, who are not being treated with a medicine that interferes with glucose metabolism, who have no allergies to soy, do not have preeclampsia, are not taking antibiotics, and are not taking proton pump inhibitors.

### Symptom and Laboratory Evaluation

Glycemic control is evaluated during treatment and 8-12 weeks following delivery. Stool analysis before treatment is initiated, during treatment and after 8-12 weeks after delivery.

### Study Design

Women selected for participation are diagnosed with gestational diabetes between 24 and 28 weeks of pregnancy that is resolved after 10 days of diet and exercise. The subjects are encouraged to consume either Activated Soy Pod Fiber or Placebo within 1 hour prior to 2 meals each day with ingestion of the test agent being mandatory prior to the 3^{rd} meal in addition to dietary advice.

Women are given an 8 week supply of ether Activated Soy Pod Fiber or Placebo at the onset and are instructed to consume the entire 180 ml volume containing either formula within 1 hour prior to either meals 1 and 2, as well as another 180 ml volume containing either formula within 1 hour prior to meal 3.

Subjects measure fasting blood glucose each morning by finger stick and report the values weekly during office visits. Comparison of the treatment group to the placebo group are made from 2-3 weeks of treatment and at 8-12 weeks following delivery.

### Outcome

This study is designed to exemplify that Activated Soy Pod Fiber and not Placebo:

1) Improves glucose regulation until term;
2) Has no serious adverse events;
3) Does not alter rates of caesarean section, preterm delivery, neonatal mortality, number of neonatal and maternal trauma related to delivery, number of days of hospitalization
2) Shifts stool profile to one representative of a healthy GI microbiome
n) Decreases stool pH;
o) Increases stool SCFA.
p) Increased stool lactoferrin
q) Decreased stool white blood cells
r) Decreased stool mucus
s) Increased stool secreted immunoglobulin A
t) Increased stool anti-gliadin secreted immunoglobulin A
u) Decreased stool pathogenic bacteria
v) Decreased stool yeast, fungi, and parasites
w) Increased stool triglycerides
x) Decreased stool branched chain fatty acids
y) No change in stool long chain fatty acids
z) Increased stool cholesterol.

### Example 10

### Production of Activated Soy Pods

Thirty (30) soy plants of a variety used for edamame (IA2032) were planted and grown in a greenhouse. Twenty (20) pods were harvested from 3 plants at the reproductive stage 6 (R6) or when the green pod contains seeds (beans) that have filled the pods. The pods were placed in a plastic bag and carried to a laboratory. Five (5) pods were transferred into a 50 ml centrifuge tube that was filled with Millipore water. A top was placed on the tube and the tube was inverted several times to wash the pods. The water was drained and the rinse was repeated. Three other sets of 5 pods were washed in a similar manner.

All 20 pods were thinly sliced by placing pod containing seeds vertically in the food pusher of a food processor that is modified with a 20 ml syringe in the center. The pod is placed into the syringe, which holds the pod vertical and delivers it to the slicing blade about 2 mm from the cutting surface. The food processor (KitchenAid® Model KFP720WH1) using disc slicing attachment was turned on slicing the pods and seeds into thin cross sections. The sliced pod tissue was transferred into a four 150 mm Petri dishes containing S & S Blue Ribbon #589 filter paper that is presoaked with 6 ml of Millipore water.

The lids of the Petri dishes were removed and the tissues were exposed to UV-B raditation using an UVitron Sunray 600, uv flood lamp (310 W/m2). The exposure was set for 2.0 min and the lids were replaced. Tissue from 1 Petri dish was immediately transferred to a 50 ml centrifuge tube and placed in a freezer at −80 C. These samples represent 0 h of incubation. The other Petri dishes containing sliced pods were placed into a sealed desiccator on a platform to isolate them from a dish of saturated potassium chloride below the platform to fix the humidity at 83%. The desiccator was placed in the dark.

After incubation for 24 h, 48 h or 72 h at 22.5 C the tissue was transferred into a 50 ml centrifuge tube and then stored at −80 C. The centrifuge lids were removed and the tubes were covered with Chem Wipe tissue that was held in place by a rubber band. The tubes were placed into a freeze dryer overnight.

Dried tissue was milled using a Glen Mills mill with a 1.0 mm screen. The dried tissue was transferred into hopper of the mill and powder was collected. The mill screen was changed to 0.5 mm size and the previously milled material was added to the hopper and milled to produce a fine powder.

In order to measure soy compounds activated by this process high performance liquid chromatography (HPLC) was performed. 0.2 g powder was weighed into a 1 ml microfuge tube. 1 ml methanol was added and the tubes were sonicated in a water bath with water level adjusted just so the vials float After 1 h of sonication the microfuge tubes were centrifuged at high speed for 5 min. The supernatant was transferred onto the filter of a 1 ml centrifuge filter and centrifuged for 5 min to remove fine particles. That filtrate was transferred to an autosampler vial for HPLC analysis (HPLC analyses were performed on a Waters 2695 combined with a Waters UV-visible 2996 photodiode array detector (Waters Associated, Milford, Mass.).

Soy compounds were separated using a Luna II C18 reverse phase column (4.6×250 mm; 5 μm; Phenomenex, Torrance, Calif.). A guard column containing the same packing was used to protect the analytical column. Solvent A was 0.1% acetic acid in water and solvent B was 100% acetonitrile.

A 30 μl volume of sample was injected and the HPLC was programmed with a flow rate of 1.0 ml/min using 15% B for 8 min, then 58% B in 50 min, then 90% B in 10 min followed by holding at 90% B for 10 min. The spectra was collected between 220 and 400 nm by a photodiode array detector.

### Data

FIG. 10 is a HPLC profile demonstrating species of soy compounds observed without incubation (0 h incubation) and new peaks of UV absorption with incubation for up to 72 h. Incubation is required for the enzyme systems in the plant tissue to process new molecules in response to physical injury (slicing) and UV-B radiation. In particular, peaks identified at 72 h represent glyceollin III (peak 13), glyceollin II (peak 14) and glyceollin I (peak 15). Also identified is coumestrol (peak 9) and glycinol (peak 4). The unknown peaks are being identified. It is clear from these data that such processing is a useful means of activating soy pod tissue to produce bioactive molecules.

FIG. 11 shows quantification of 3 glyceollin species produced. One bar represents summation of the 3 glyceollin species that is as high as 1.5 mg/g powder if the incubation is performed for 96 h. The most abundant species are glyceollin III and glyceollin I, which together represent about 80% of glyceollin generated.

### INCORPORATION BY REFERENCE

All references, articles, publications, patents, patent publications, and patent applications cited herein are incorporated by reference in their entireties for all purposes.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising: isolated plant tissue having at least 0.25 mg glyceollin content per gram of plant tissue.

2. The composition of claim 1, wherein the isolated plant tissue is soy pod tissue.

3. The composition of claim 2, wherein the combined total content of one or more glyceollins is at least 1 mg per gram of soy pod tissue.

4. The composition of claim 2, wherein the combined total content of one or more glyceollins is at least 5 mg per gram of soy pod tissue.

5. The composition of claim 2, wherein the combined total content of one or more glyceollins is at least 10 mg per gram of soy pod tissue.

6. The composition of claim 2, wherein the combined total content of one or more glyceollins is at least 100 mg per gram of soy pod tissue.

7. The composition of claim 2, wherein the soy pod tissue contains soluble and insoluble dietary fiber.

8. The composition of claim 1, formulated for oral delivery.

9. The composition of claim 1, formulated as a food product.

10. The composition of claim 1, formulated as a powder.

11. The composition of claim 10, wherein the powder is made from soy pod tissue.

12. A method of treating a subject suffering from obesity, comprising:
orally administering to the subject a composition according to claim 1.

13. A method of treating a subject suffering from diabetes, comprising:
orally administering to the subject a composition according to claim 1.

14. A method of modifying the gastrointestinal microbiome of a subject, wherein the gastrointestinal microbiome of the subject includes a first population of bacteria that process fat and protein, and a second population of bacteria that ferment carbohydrate and produce increases in small chain fatty acids, comprising:
administering to the subject a composition according to claim 1,
wherein said composition is administered in an amount effective to shift the relative abundance of the first population of bacteria and the second population of bacteria in the gastrointestinal tract of the subject.

15. The method of claim 14, wherein the first population comprises the genus of Ruminococcaceae and the second population comprises the genus of Blautia.

16. A method of modifying the gastrointestinal microbiome of a subject, comprising:
administering to a subject whose microbiota taxa is determined to contain a level of Blautia below 5% abundance,
a composition according to claim 1, in an amount effective to increase Blautia levels to at least 20% abundance.

17. A method for treating gastrointestinal dysbiosis in a subject, comprising:
orally administering to the subject an effective amount of a composition according to claim 1.

18. A method of manufacturing a powder comprising soy pod dietary fiber and one or more glyceollins, comprising:
a. obtaining soy pod tissue;
b. slicing or mincing the soy pod tissue;
c. drying the soy pod tissue; and
d. pulverizing the soy pod tissue into a powder.

19. The method of claim 18, further comprising: adding one or more glyceollins to the soy pod tissue.

20. A powder manufactured according to the method of claim 18.
